(19) United States
(12) Patent Application Publication
(10) Pub. No.: US 2005/0075315 A1
(43) Pub. Date: Apr. 7, 2005

(54) Antitumor agent

(75) Inventors: Hiroshi Takeyama, Tokyo (JP); Tsuneya Ohno, Tokyo (JP)

Correspondence Address: SUGHRUE MION, PLLC, 2100 PENNSYLVANIA AVENUE, N.W., SUITE 800, WASHINGTON, DC, 20037, US

(73) Assignees: FUSO PHARMACEUTICAL INDUSTRIES, LTD; TSUNEYA OHNO

(21) Appl. No.: 10/611,902

(22) Filed: Jul. 3, 2003

(30) Foreign Application Priority Data
Nov. 25, 2002 (JP) 2002-341415

(51) Int. Cl. A61K 31/727; A61K 31/045; A61K 31/375

(52) U.S. Cl. 514/56; 514/474; 514/730

(57) ABSTRACT

The present invention is meant to provide a novel use of benzyl alcohol for an antitumor agent guaranteed to have sufficient safety. The present inventors have made a study on the possible action of benzyl alcohol on a tumor cell to develop the use and a treatment method thereby. As a result, it has been found out that the local administration to a cancer cell site can promote the cancer cell to be separated off very efficiently and specifically. Thus, the usefulness for an antitumor agent is confirmed. Moreover, the combination with an antioxidant has been found out to increase the effect.

The application claims priority from Japan Patent Application NO.2002-341415 which is incorporated herein by reference.

### FIELD OF THE INVENTION

The present invention relates to a novel antitumor agent and a method for treating a tumor, and more particularly to a novel use of benzyl alcohol.

### BACKGROUND OF THE INVENTION

Benzyl alcohol (hereinafter, sometimes abbreviated as “BA”) is described to be a low toxic local anesthetic in the Japanese Pharmacopoeia, and shows a local anesthetic action when administrated in a low concentration of the aqueous solution, while a local stimulative action when administrated in a high concentration. Benzyl alcohol also has an antibacterial action and is reported to have a phenol coefficient of 0.4 on Sarmonella typhii, 0.6 on Escherichia coli, and 0.5 on Staphylococcus aureus. Benzyl alcohol is converted to benzoic acid in body and excreted as hippuric acid. As for application, a 10% ointment or a lotion containing ethanol, water and benzyl alcohol in their equal amounts is used for an antipruritic because of its local anesthetic action and disinfection. Benzyl alcohol also is dropped to a dental nerve or a dental pocket for an analgetic in toothache. Benzyl alcohol also is added in an injection solution at a rate of 1-4% in order to mitigate a pain brought about by subcutaneous or intramuscular injection. At present, BA is seldom used alone for a local anesthetic, but often used for an additive solvent in an intramuscular or a subcutaneous injection agent. BA is generally used in the 0.9% aqueous solution (W/V % in a physiological saline) and can be administered at a dose of 200-300 mg/day.

BA is known to be a free radical scavenger because it works to eliminate cytotoxic active oxygen which produces a carcinogen such as nitrosoamine formed in body by the uptake of mixed foods (for example, a combination of a burnt fish or meat piece with a certain vegetable), and is thought to have a carcinogenesis inhibiting function. However, there is found no report indicating that BA has a direct antitumor effect on cancer as long as the review made by the inventors.

### SUMMARY OF THE INVENTION

An object of the present invention is to provide a novel use of benzyl alcohol as a sufficiently safe antitumor agent.

The present inventors have made various studies on the possible action of benzyl alcohol on a tumor cell to develop the use and a treatment method thereby. As a result, it has been found out that the local administration of BA to a cancer cell site can promote the cancer cell to be separated off very efficiently. Thus, the usefulness for an antitumor agent is confirmed and moreover, the combination with an antioxidant has been found out to increase the effect. These findings complete the invention.

Namely, the invention includes:
1. A method for treating a tumor comprising the step of administrating a composition containing benzyl alcohol as the active ingredient.
2. A method for treating a tumor comprising the step of external administrating a composition containing benzyl alcohol as the active ingredient.
3. A method for treating a tumor comprising the step of perorally or parenterally administrating a composition containing benzyl alcohol as the active ingredient.
4. A method for treating a tumor comprising the step of administrating a composition containing benzyl alcohol as the active ingredient in combination with heparin and/or vitamin C.
5. A method for treating a tumor comprising the step of administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.
6. A method for treating a tumor comprising the step of external administrating a composition containing benzyl alcohol as the active ingredient in combination with heparin and/or vitamin C.
7. A method for treating a tumor comprising the step of perorally or parenterally administrating a composition containing benzyl alcohol as the active ingredient in combination with heparin and/or vitamin C.
8. A method for treating a tumor comprising the step of external administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.
9. A method for treating a tumor comprising the step of perorally or parenterally administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.
10. A method for treating a tumor comprising the step of administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.
11. A method for treating a tumor comprising the step of external administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.
12. A method for treating a tumor comprising the step of perorally or parenterally administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.
13. A method for treating a tumor according to any of above 1 to 12, wherein said benzyl alcohol is administrated at a dose necessary and sufficient to cause a tumor cell to fall in necrosis.
14. A method for treating a tumor according to any of above 1 to 12, wherein said benzyl alcohol is administrated at a dose necessary and sufficient to cause a tumor cell to fall in necrosis, said dose being determined as an amount necessary and sufficient by using separation of a tumor cell from a normal cell as a marker.
15. A method for treating a tumor according to any of above 1 to 12, wherein said benzyl alcohol is administrated at a dose necessary and sufficient to cause a tumor cell to fall in necrosis, said dose being 1 mg-50 mg/tumor volume (cm³).
16. A method for treating a tumor according to any of above 1 to 12, wherein said benzyl alcohol is administrated at a dose of 1 mg-50 mg/tumor volume (cm³) as an amount necessary and sufficient to cause a tumor cell to fall in necrosis, said dose being determined as an amount necessary and sufficient by using separation of a tumor cell from a normal cell as a marker.
17. A method for treating a tumor according to any of above 1 to 12, wherein said administration is carried out by using an aqueous 0.1-5% benzyl alcohol solution.

### BRIEF DESCRIPTION OF DRAWINGS

FIG. 1 shows the result of a STKM cell by the calorimetric assay;

FIG. 2 shows the result of a STKM cell by the Thymidine incorporate assay;

FIG. 3 is the inverted microscopic photograph of a STKM cell in vitro. Left: with physiological saline added, and Right: with BA added;

FIG. 4 is the inverted microscopic photograph of a H-E dyed STKM cell in vivo, with physiological saline added;

FIG. 5 is the inverted microscopic photograph of a H-E dyed STKM cell in vivo, with BA added;

FIG. 6 shows the result of gel electrophoresis of DNA Laddering in vitro;

FIG. 7 is the inverted microscopic photograph of a STKM cell by the TUNEL assay;

FIG. 8 shows the result of measured Caspase-3 activity;

FIG. 9 shows the result of measured Caspase-8 activity; and

FIG. 10 shows the result of a normal cell (Huvec, WI38) by the calorimetric assay.

### BEST MODE FOR CARRYING OUT THE INVENTION

In the present invention, the active ingredient is benzyl alcohol. The properties, the determination method, the production method, the pharmaceutical effect, and the application are detailed in the Japanese Pharmacopoeia and are omitted. The formulations are also detailed there. Antitumor in the invention is meant to deal with a wide variety of cancer cells, preferably to a cell epidermal carcinoma. Good results are obtained on stomach cancer, breast cancer, colon cancer, rectum cancer, cecum cancer, large bowel cancer, cervical cancer, thyroid gland cancer, pancreas cancer, and malignant melanoma, etc.

The formulation may be for a local administration or a systemic administration and preferably is for a local administration, though not limited particularly. The dosage form is not limited particularly and may be peroral or parenteral such as dermal, intramuscular, and the like. Any forms are applicable and may be selected depending on the kind and the site of a tumor.

In the invention, BA administrated alone can represent a sufficient antitumor effect, but more preferably the combination with an antioxidant can provide a good result in the infiltration into and the separation of a cancer cell. The antioxidant includes, for example, vitamin A, vitamin C and heparin. The addition amount is, for example, 0.1-10 times 1 part of BA by weight.

In the case of an injection liquid for example, a formulation is adjusted to have a BA content of 1-5 (w/v) %. In the invention, the dose of the antitumor agent depends upon the administration route and is difficult to define as a generic amount, but can be determined using necrosis of a tumor cell or separation of a tumor cell from a normal cell as a marker and administered by such an amount necessary and sufficient to cause a tumor cell to fall in necrosis or to separate from a normal cell. The dose of BA may be 1-50 mg/tumor volume (cm³), preferably 1.5-30 mg/tumor volume (cm³), and more preferably 2.3-18.86 mg/tumor volume (cm³). Benzyl alcohol is suitably administered by using an aqueous 0.1-5%, preferably 0.76-4% (w/v) solution. The administration of one to a few times a day, every day or every second day for 10 days to a few months can attain the above separation effect.

The treatment method for destructing a tumor cell in human body by the local administration of dehydrated ethanol (100%) to the tumor is currently applied to liver cancer as the PEIT (Percutaneous Ethanol Injection) therapy. In this case, the tumor volume is measured by CT or ultrasonic analysis, and a value of “length×width×height” multiplied by Π/6, that is, “Π/6×length×width×height” is generally used as a dose. The method can also be used for calculating the dose of BA in the local administration to a tumor according to the invention.

The calorimetric assay as described in the examples below can be employed for assaying the adhesiveness of a cell, and the thymidine incorporate assay can be employed for assaying the mortality of a cell. These two assays are general methods for determining the behavior of a tumor cell. For the colorimetric assay, see Enhancer sequences of DF3 gene regulate expression of the herpes simplex virus thymidine kinase gene and confer sensitivity of human breast cancer cell to ganciclovir: Manome Y, Abe M, Hagen F. M, Fine A. H, and Kufe W. D, Cancer Research 54:5408-5413, 1994. For the thymidine incorporate assay, see Meneki Jikken Sousa-ho II (edited by S. Uda, S. Konda, Y. Honsho, T, Hamaoka, 773-774, Nanko-do, 1995).

The DNA Laddering assay, the TUNEL assay and the Caspase assay as described in the examples below are general methods for determining whether a cell arrives at death through an apoptosis route or not. For the DNA Laddering assay, see Shin Apoptosis Jikken-ho (edited by K. Tsujimoto, S. Tone, T. Yamada, 59-66, Yodosya, 1999). For the TUNEL assay, see Shin Apoptosis Jikken-ho (edited by K. Tsujimoto, S. Tone, T. Yamada, 67-74, Yodosya, 1999). For the Caspase assay, see Shin Apoptosis Jikken-ho (edited by K. Tsujimoto, S. Tone, T. Yamada, 198-200, Yodosya, 1999).

### EXAMPLES

The invention will be described more specifically with reference to the following examples. They must be noted to help understanding the invention concretely, and not to limit the scope of the present invention.

### Example 1

### The Antitumor Effect of BA on a Stomach Cancer Cell in vitro

To 1×10⁶ cells of a stomach cancer cell line (STKM), BA was added by such proportional concentrations as 1, 1.5, 2.0, 2.5 and 5 mg/ml. An equal amount of the physiological saline was added to prepare a control sample. All the samples were incubated at 37° C. for 48 hrs. The shape of the cell was studied through an inverted microscope. The adhesiveness and the mortality of the cell were measured by the calorimetric assay and the thymidine incorporate assay.

(1) The Cell Adhesiveness Test by the Calorimetric Assay:

100% gulutalaldehyde was added to each of samples by a ¼ the medium amount (250 μl), then left to stand at a room temperature for 15 min. After washing, 250 μl of 0.05% Methylene Blue in PBS was added and then left to stand at a room temperature for 15 min. After washing, 250 μl of 0.33N HCl was added, followed by leaving to stand at a room temperature for 15 min and measuring the OD (600 nm).

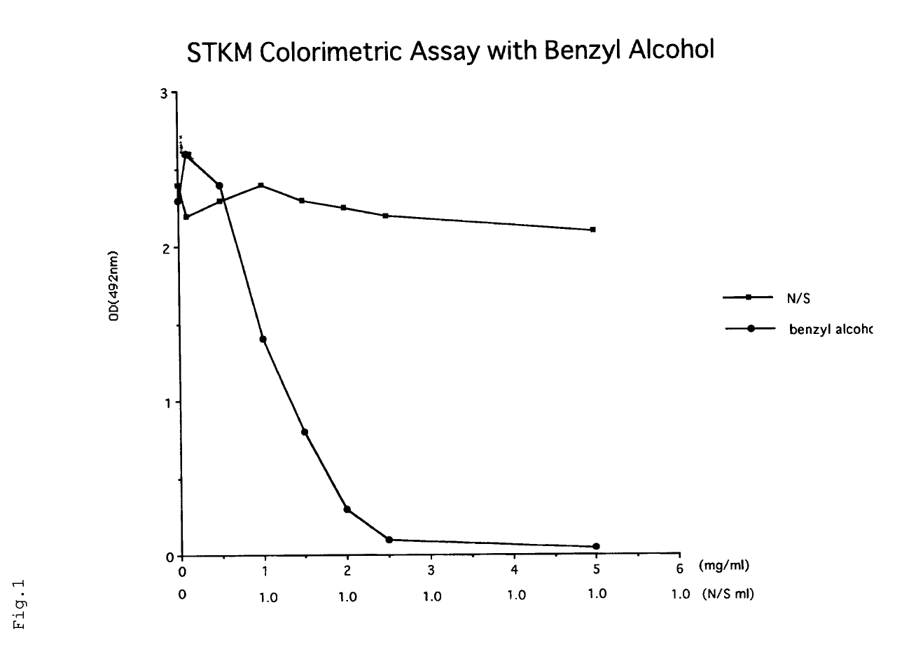

By BA was added proportionally in concentration, and the cell adhesiveness began with about 1.5 mg/ml of BA to decrease proportionally (FIG. 1).

(2) The Cell Mortality Test by the Thymidine Incorporate Assay:

Thymidine-methyl-³H (0.37 kBq/10 μl, ICN, Biochemicals, Inc. Irvine Calif.) was added to the 1×10⁶ cells followed by incubating at 37° C. for 6 hrs. The resultant was terminated to react by 50% TCA and filtered by the GF/C glass filter (10 mm diameter) (Whatman, Maidstone, England). The filter and the precipitate were washed five times with 5% TCA. 7 ml of the scintigram cocktail (Optiphase HiSafe 2, Wallac Scintillation Products, Turku, Finland) was then added to the filter and the precipitate to count the amount of radiation for 1 min by the Beckman LSLiquid scintillation counter (Beckman, Alvertville, Minn.).

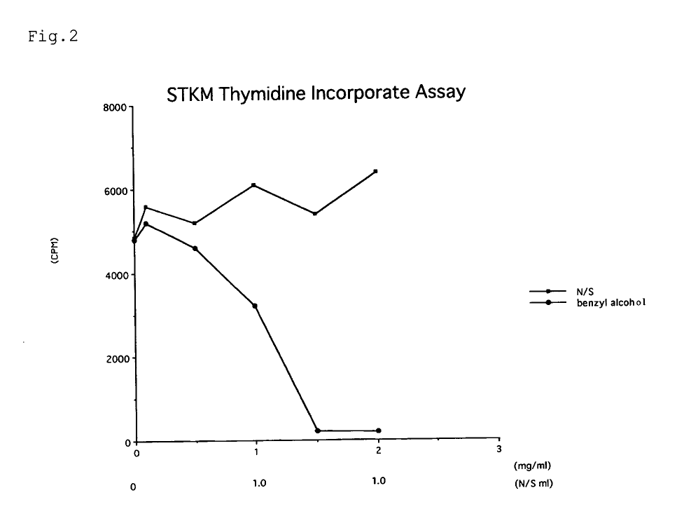

The cell mortality began with about 1 mg/ml of BA to increase in proportion to the BA concentration (FIG. 2) as was the case with adhesiveness.

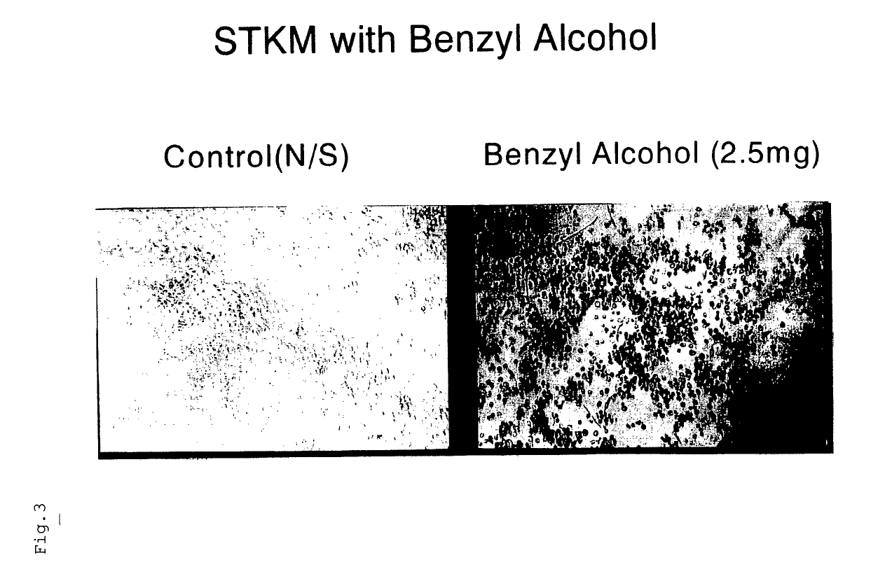

One ml of BA of 2.5 mg/ml was added to 1×10⁶ cells followed by incubating at 37° C. for 48 hrs to get the image as shown in FIG. 3. The equal amount of physiological saline was similarly added, followed by incubating in the same way to get the control. The BA sample image shows that the cell shape changed from a spindle to a circle and got swollen. The nucleus also got remarkably blackened. The above result confirms that a stomach cancer cell arrived at death in vitro.

The result in Example 1 proves that BA has an antitumor effect on a stomach cancer cell in vitro, and that the cell mortality begins with about 1.5 mg/ml of BA to increase depending on the BA concentration.

### Example 2

### The Antitumor Effect of BA on a Stomach Cancer Cell in vivo

1×10⁸ cells of a stomach cancer cell line (STKM) were subcutaneously injected in the back of a nude mouse. When a tumor grew to have a diameter of 5 mm or more after 8 weeks, BA was administrated to the tumor or around the tumor by 4 mg/0.5 ml twice a week for 4 weeks. For a control, 0.5 ml of physiological saline was locally injected to the tumor. The tumor diameter was measured. The tumor tissue was taken out and subjected to the H-E dyeing to observe with a microscope.

A stomach cancer cell line (STKM) was implanted in a living body. When a tumor grew to have a diameter of 5 mm or more, BA was administrated by 4 mg/0.5 ml twice a week for 4 weeks. At the point of the 16 mg administration (2 weeks), the tumor contracted in diameter compared with the control. The tumor diameter decreased to the ½ or less after the 32 mg administration (4 weeks).

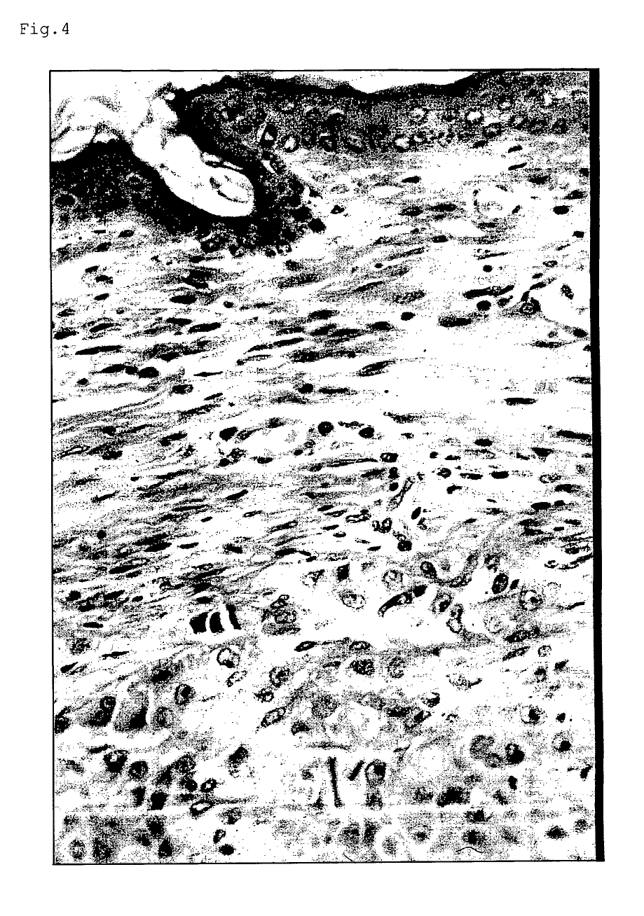

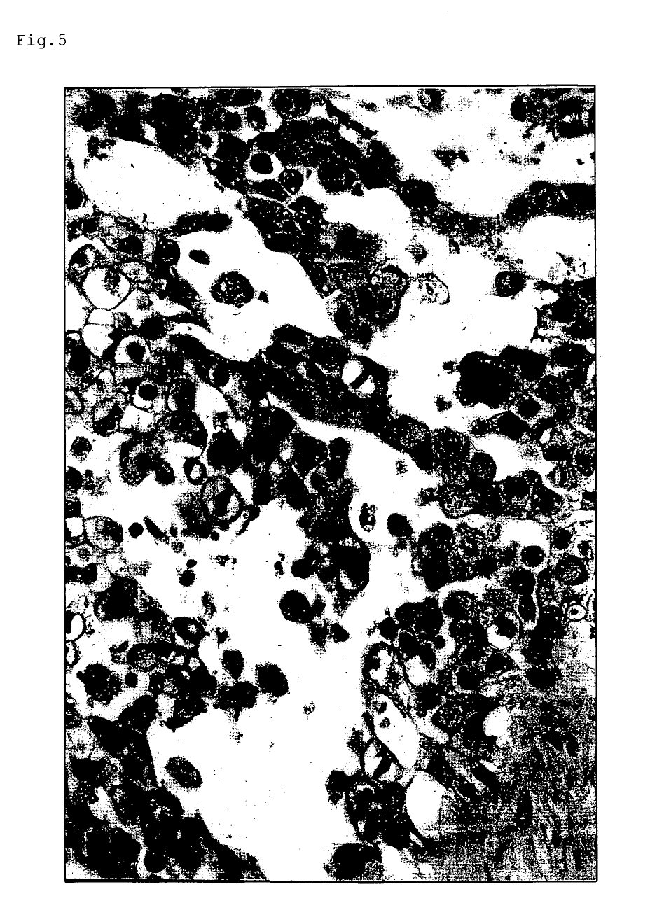

FIGS. 4 and 5 show the images obtained by extracting the tumor after 4 weeks of administration, fixing with 10% formalin, slicing to 3 μm pieces and H-E dyeing. FIG. 4 is a control image after the administration of physiological saline, and reveals that the tumor cells grew up to the skin to contact closely each other, and that both the cytoplasm and the nucleus were firm. FIG. 5 is a test image after the administration of BA, and reveals that the cells were scattered sparsely with the swollen cytoplasm, appearance of the inclusion bodies and contraction of the nucleus. The above result confirms that the stomach cancer cell arrived at death in vivo.

The result in Example 2 proves that BA has an antitumor effect on a stomach cancer cell in vivo.

### Example 3

### The Cell Mortality Effect of BA

For a cell group capable of being guided to the cell mortality, it was determined that through which route, apoptosis or necrosis, the cell arrived at death. Namely, BA was added to the cell at a certain concentration or more necessary to guide to the cell mortality, and the cell was collected after confirmation of the change in shape. Then, the DNA Laddering assay with gel electrophoresis to investigate the DNA fragmentation and the TUNEL assay with the cell dyeing were carried out.

The DNA Laddering Assay:

2 ml of BA of 2.5 mg/ml was added to 1.5×10⁶ cells (STKM) followed by incubating at 37° C. for 48 hrs to collect the cells. The collected cells were centrifuged and added in 100 μl of lysis buffer (10 mM EDTA, 50 mM Tris-HCl, pH 8.0, 0.5% SDS, 0.5 mg/ml Proteinase K), followed by incubating at 5° C. for 3 hrs. Then, 100 μl of loading buffer (10 mM EDTA, 1% w/v low melting agarose, 0.25% bromo phenol blue, 40% sucrose) was added to give a sample, which was subjected to electrophoresis (37 V, over night) with 2% agarose gel in TAE buffer.

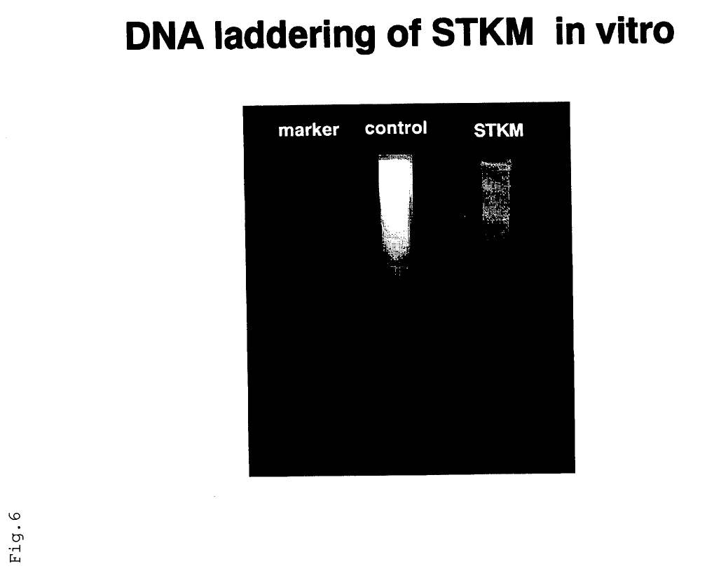

No laddering was recognized also in STKM (a stomach cancer cell line) to which 2 ml of BA of 2.5 mg/ml was added (FIG. 6).

The TUNEL Assay:

2 ml of BA of 2.5 mg/ml was added to 1.5×10⁵ cells (STKM) on a slide glass, followed by incubating at 37° C. for 48 hrs and fixing with 1% paraformaldehyde in PBS at a room temperature for 10 min. For a control, the equal amount of physiological saline was added. Then, the Apoptag In Situ Apoptosis Detection kits (Intergen, N.Y., US) was applied to the sample piece. The application was briefly as follows:
1. 3% hydrogen peroxide in PBS was added to a piece at a room temperature for 5 min, followed by
2. reacting with the Working strength TdT enzyme at 37° C. for 1 hr,
3. reacting with the anti-Digoxigenin conjugate at a room temperature for 30 min,
4. reacting with the Working strength peroxidase substrate (room temperature, 3-6 min), and
5. reacting with 0.5% methyl green (room temperature, 10 min) for counter staining.

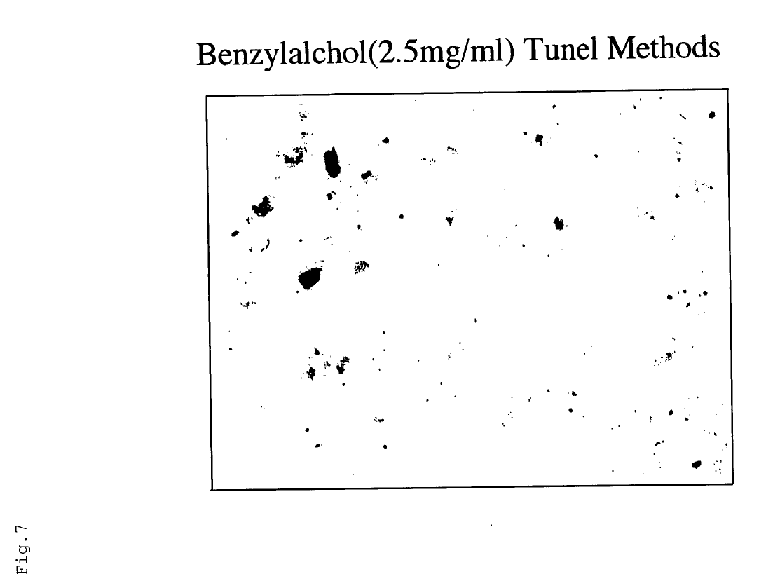

The cell line added with 2 ml of BA of 2.5 mg/ml showed no Digoxigenin dyeing image in the nucleus (FIG. 7).

Caspase-3, Caspase-8 activity assay:

To 1.5×10⁶ cells, 2 ml of BA of 2.5 mg/ml for a test sample, the equal amount of physiological saline for a negative control sample, and Arac 1×10⁻⁵M (Kirosaito, Nippon Shinyaku) for a positive control sample were added respectively, each of which was followed by incubating at 37° C. for 48 hrs to recover the cells. Then, the CPP32/Caspase-3, -8 Colorimetric Protease Assay kit (MBL Co.) was applied to the samples. The application was briefly as follows:
1. 0.5 ml of cell lysis buffer was added to the recovered cells to react at a room temperature for 10 min, followed by
2. sonication (30 sec×2, Branson, SONIFIER 250),
3. centrifugal separation (10,000 rpm, 3 min, HITACHI, himac CF15D),
4. recovering the supernatant,
5. adding 50 μl of reaction buffer and 5 μl of the activity assay substrate (IETD-pNA) to 50 μl of the supernatant to react at 37° C. for 1 hr, and
6. measuring the fluorescence intensity (excitation wavelength 400 nm, fluorescence wavelength 505 nm).

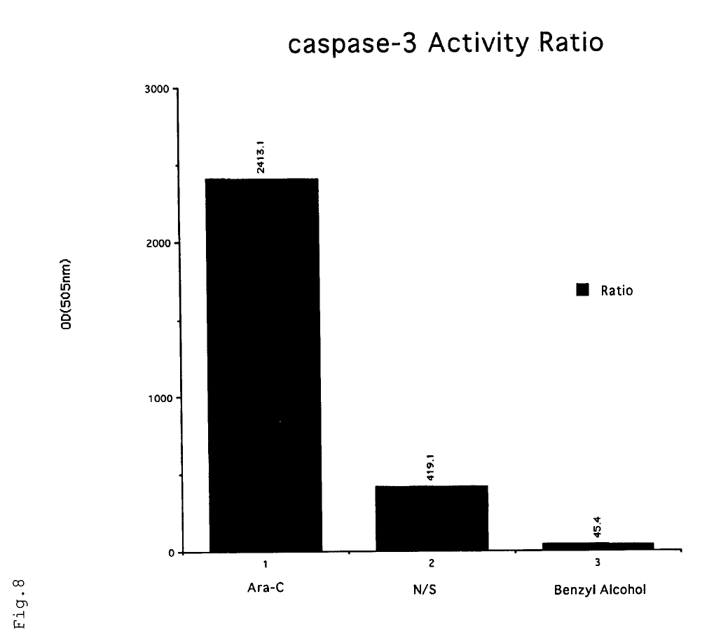

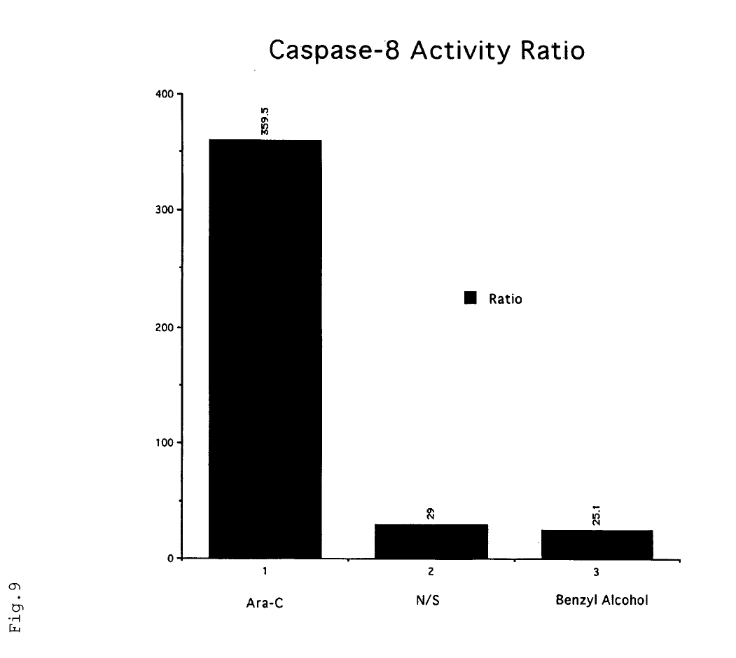

In the Caspase-3 active assay, the value of the BA-added cell had 45.4 (RFI/well/Hr), the said value being significantly lower than that of the positive control (p<0.001). Similarly, in the Caspase-8 activity, the value of BA-added cell was significantly lower than that of the positive control. The value was almost equal to that of the negative control (FIGS. 8, 9).

The result in Example 3 proves that the BA administration effects the cell to arrive at death through a necrosis route.

### Example 4

### The Effect of BA Administration to a Normal Cell

In order to confirm that the BA administration effects specifically a cancer cell to arrive at death, the colorimetric assay was carried out on a normal cell, that is, an umbilical cord cell line (Huvec) and a lung cell line (WI38), in the same way as in Example 1. The stomach cancer cell line (STKM) was used for a control.

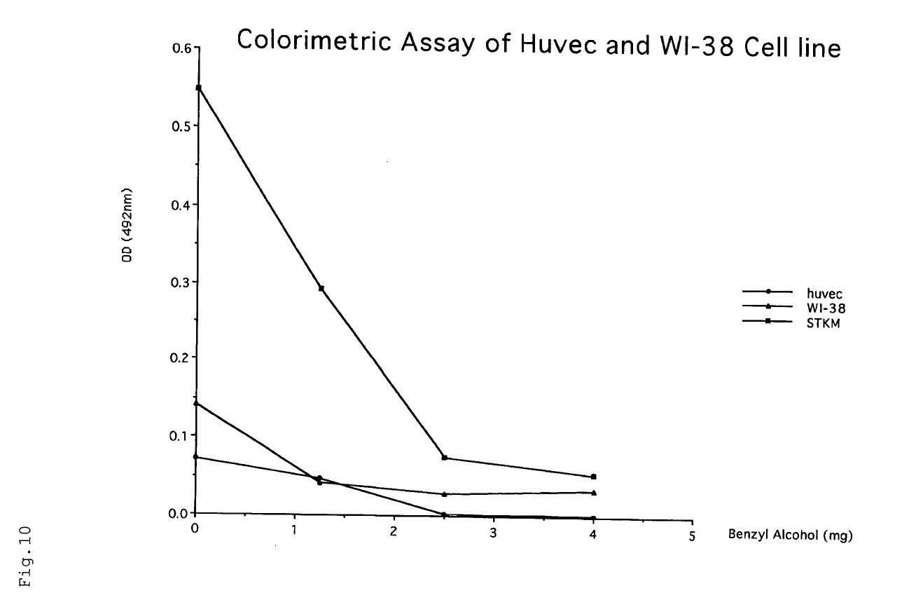

The BA administration still caused both the Huvec and the WI38to arrive at death depending on the BA concentration, as found in the cancer cell line, but with a lower effect compared to the STKM. Though the initial numbers of Huvec and WI38were about 1/10 of that of STKM, the WI38was more resistant to BA than the Huvec (FIG. 10).

The result in Example 4 proves that the BA administration effects a cancer cell to arrive at death more strongly than a normal cell.

### Example 5

The tests were carried out on a breast cancer cell line (MCF-7, BSMZ), a large bowel cancer cell line (DLD, LOVO), a thyroid gland cancer cell line (SW1736), and a pancreas cancer cell line (PA-1) in the same ways as in Examples 1, 2, and 3. As the result, the cell mortality began with about 1.5 mg/ml of BA to increase in proportion to the BA concentration, same as adhesiveness thereof. Furthermore, it was confirmed that the cells did not arrive at death through an apoptosis route, but through a necrosis one.

The administration of benzyl alcohol according to the invention, which is guaranteed to have safety, can achieve an antitumor effect. Thus, benzyl alcohol according to the invention can provide an effective means for treating a cancer.

Thus, the present invention can successfully provide a novel use of benzyl alcohol for an antitumor agent guaranteed to have sufficient safety.

1. A method for treating a tumor comprising the step of administrating a composition containing benzyl alcohol as the active ingredient.

2. A method for treating a tumor comprising the step of external administrating a composition containing benzyl alcohol as the active ingredient.

3. A method for treating a tumor comprising the step of perorally or parenterally administrating a composition containing benzyl alcohol as the active ingredient.

4. A method for treating a tumor comprising the step of administrating a composition containing benzyl alcohol as the active ingredient in combination with heparin and/or vitamin C.

5. A method for treating a tumor comprising the step of administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.

6. A method for treating a tumor comprising the step of external administrating a composition containing benzyl alcohol as the active ingredient in combination with heparin and/or vitamin C.

7. A method for treating a tumor comprising the step of perorally or parenterally administrating a composition containing benzyl alcohol as the active ingredient in combination with heparin and/or vitamin C.

8. A method for treating a tumor comprising the step of external administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.

9. A method for treating a tumor comprising the step of perorally or parenterally administrating a composition containing benzyl alcohol as the active ingredient, wherein a tumor associated with said composition is breast cancer, large bowel cancer, thyroid gland cancer, colon cancer, cecum cancer, cervical cancer, malignant melanoma, pancreas cancer, or stomach cancer.

10. A method for treating a tumor comprising the step of administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.

11. A method for treating a tumor comprising the step of external administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.

12. A method for treating a tumor comprising the step of perorally or parenterally administrating a composition containing benzyl alcohol as the active ingredient in combination with heparin and/or vitamin C, wherein a tumor associated with said composition is breast cancer, large bowel cancer, thyroid gland cancer, colon cancer, cecum cancer, cervical cancer, malignant melanoma, pancreas cancer, or stomach cancer.

13. A method for treating a tumor according to any of claim 1 to 12, wherein said benzyl alcohol is administrated at a dose necessary and sufficient to cause a tumor cell to fall in necrosis.

14. A method for treating a tumor according to any of claim 1 to 12, wherein said benzyl alcohol is administrated at a dose necessary and sufficient to cause a tumor cell to fall in necrosis, said dose being determined as an amount necessary and sufficient by using separation of a tumor cell from a normal cell as a marker.

15. A method for treating a tumor according to any of claim 1 to 12, wherein said benzyl alcohol is administrated at a dose necessary and sufficient to cause a tumor cell to fall in necrosis, said dose being 1 mg-50 mg/tumor volume (cm³).

16. A method for treating a tumor according to any of claim 1 to 12, wherein said benzyl alcohol is administrated at a dose of 1 mg-50 mg/tumor volume (cm³) as an amount necessary and sufficient to cause a tumor cell to fall in necrosis, said dose being determined as an amount necessary and sufficient by using separation of a tumor cell from a normal cell as a marker.

17. A method for treating a tumor according to any of claim 1 to 12, wherein said administration is carried out by using an aqueous 0.1-5% benzyl alcohol solution.
